(19) United States
(12) Patent Application Publication
(10) Pub. No.: US 2006/0216347 A1
(43) Pub. Date: Sep. 28, 2006

(54) Tablet punches and methods for tableting

(76) Inventors: Federico Stroppolo, Aldesago (CH); Sandro Lepori, Oggio (CH); Franco Ciccarello, Via La Roggia (CH); Rita Milani, Via Ganna (CH)

Correspondence Address: James V Costigan;Hedman & Costigan, 1185 Avenue of the Americas, New York, NY, 10036-2646, US

(21) Appl. No.: 10/567,055

(22) PCT Filed: Aug. 16, 2004
(86) PCT No.: PCT/IB04/02668

§ 371 Date: Mar. 6, 2006

Related U.S. Application Data
(60) Provisional application No. 60/497,580, filed on Aug. 25, 2003.

(51) Int. Cl. A61K 9/20; B27N 3/00

(52) U.S. Cl. 424/464; 264/109; 249/134; 425/412

(57) ABSTRACT

A tablet punch having a punch cup having a non-stick polymeric coating which may comprise a fluoropolymer such as polytetrafluoroethylene.

### FIELD OF THE INVENTION

The present invention relates to non-stick tablet punches and to methods for tableting using non-stick tablet punches. More specifically, the invention relates to tablet punches tipped with a non-stick polymer layer or a polymeric head and methods for tableting using such punches.

### BACKGROUND OF THE INVENTION

Solid dosage forms such as tablets and hard gelatin capsules are used widely for the administration of pharmaceuticals because of their relative ease to handle, transport and administer. Such dosage forms are usually made from compositions which include an active ingredient and one or more inert ingredients which are collectively referred to as excipients. Excipients serve to dilute the active ingredient in the composition by adding inert bulk, and can improve properties of the composition such as compressibility and processability and/or properties of the dosage form such as hardness, friability, and appearance.

Tableting presses have long been utilized to manufacture of solid forms by the compression of powders, granules and other materials referred to collectively as compression compositions. Typical tableting presses comprise at least one pair of opposing tablet punches and a die through which the punches move in a reciprocating fashion. The die is commonly made of a metal such as steel in the shape of a disc with at least one passageway provided therethrough. Each of the pair of tablet punches is also commonly metal having an elongated portion ending in a tip having a concave cup or a flat head. The tablet punches are arranged in the tablet press with the cup of each punch facing the other of its pair within the passageway of the die. At least one of the punches is capable of travel along at least a portion of the passageway. The tablet press further includes means for driving at least one of the tablet punches toward the other in order to reduce the volume of space defined by the punch cups and the wall of the die passageway to the size of the dosage form to be made thereby. Thus, a dosage form is produced in a tablet press by providing a quantity of the pharmaceutical composition to the press and engaging the driving means for relative movement of the tablet punches toward each other through the die to compress the pharmaceutical composition into the form of an oral I dosage form.

In the operation of tablet presses, and particularly high-speed tablet presses used in commercial pharmaceutical production facilities, there are a variety of technical problems which must be addressed. These problems include the proper distribution of the active ingredient dose throughout the dosage form, processing defects in the dosage form caused by capping or sticking, deficiencies in the mechanical properties of the dosage form, and other problems as described in Pharmaceutical Dosage Forms, Marcel Dekker, Lieberman & Laclunan, Vol. I (1980) at 181-182. A common result of the compression process is the production of beat. This heat builds up on the punch cups and in the die. Over a relatively short period of time, the heat build-up tends to cause the powder to stick to the faces of the punches and sides of the die. Once the powder begins to stick to the die and/or to the punch faces, the integrity of the tablet is compromised, since as the faces of the punches pull apart to eject the tablet, the tablet sticks to the punch faces and breaks, or is broken or damaged by the friction with the cylindrical walls of the die. Moreover, any powder remaining on the punches and/or die walls tends to add to the volume of powder within the die, to disadvantageously alter the internal pressure of the tablets. Tablets formed under excessive pressure may not dissolve properly, while tablets formed under insufficient pressure tend to break prematurely. These problems are well-known, and several approaches have been made.

One approach involves the use of excipients in the pharmaceutical composition such as, 10 for example, magnesium stearate, calcium stearate or stearic acid. These excipients, known variously as lubricants, diluents, coating agents, and anti-adhesive excipients, however, can present a number of disadvantages when included in a tableting composition. Most lubricants have a hydrophobic character and thus tend to inhibit or delay the disintegration and dissolution of the tablet after administration to a patient. This, in turn, can affect the bioavailability of the active ingredient to the patient and the efficacy of the dosage form. The use of lubricants can also decrease the compressibility of the pharmaceutical composition which can result in a softer, more friable dosage form. In addition, certain lubricants react chemically or physically with the active ingredient or the excipients causing discoloration or a loss in activity of the active ingredient. In an alternative approach to addressing manufacturing problems, a lubricant is applied to the punch tips to facilitate the release of the compressed form from the punches. This approach, however, requires the coordinated application and reapplication of a sufficient quantity of lubricant to the punch tips as part of the operation of the tablet press.

### REPORTED DEVELOPMENTS

U.S. Pat. No. 3,461,195 discloses a method for spraying lubrication on the punches of a single station tablet press.

U.S. Pat. No. 3,042,531 discloses a method for applying lubricant to the dies and punch faces of a commercial tablet press by first tableting a material lubricated with sodium chloride.

U.S. Pat. No. 4,047,866 discloses a tablet press which automatically applies lubricant to the die and punch faces when the force required to eject the tablet from a given die reaches a predetermined value.

### SUMMARY OF THE INVENTION

A non-stick tablet punch and a tablet press equipped with at least one pair of such non-stick tablet punches are described. The tablet punch comprises a punch cup having a non-stick polymer layer disposed on the surface of the punch cup, preferably a fluoropolymer coating, and more preferably a polytetrafluoropolymer such as PTFE film, or a non-stick head preferably comprising a fluoropolymer and more preferably a polytetrafluoropolymer such as PTFE or polytetrafluoroethylene. The non-stick coating is readily bonded to punch cups of varying sizes and shapes, and the non-stick head is readily fixed to the tablet punch. Both the tablet punch having such a non-stick coating and the non-stick head are capable of producing a compressed form with minimal or no lubricating agent present in the compression composition.

Another aspect of the present invention is directed to methods for manufacturing compressed forms such as tablets and hard gelatin capsules from a composition. The method comprises the step of by compressing a composition in a tablet press having at least one pair of opposing tablet punches comprising either punch cups having non-stick coatings adhered to the surface of the punch cups or a non-stick head fixed on the punches. The method of the present invention permits the manufacture of compressed forms such as pharmaceutical dosage forms with minimal or no lubricating agent present in the composition. The use of non-stick tablet punches minimizes manufacturing problems such as sticking and capping, and allows for the controlled manufacture of products that exhibit uniformity in size, weight and appearance.

### BRIEF DESCRIPTION OF THE DRAWINGS

FIG. 1A is a side view of the upper part of an upper tablet punch of the present invention.

FIG. 1B is a side view of the upper part of a lower tablet punch of the present invention.

FIG. 2A is a view from above the end upper tablet punch shown in FIG. 1A.

FIG. 2B is a view from above the end of the lower tablet punch shown in FIG. 1A.

FIG. 3A is a schematic side view of an upper tablet punch.

FIG. 3B is a schematic side view of a lower tablet punch.

FIG. 3C is an expanded section taken transversely through the center of the punch cup of the punch FIG. 3B.

### DETAILED DESCRIPTION OF THE INVENTION

One aspect of the invention is a non-stick tablet punch.

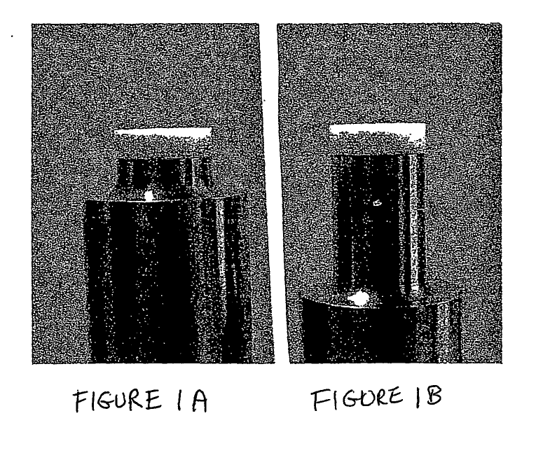

FIG. 1A shows the side of the upper part of a tablet punch for use in a high speed rotary tableting machine where a PTFE punch cup 2 is mounted on the end that is used to contact a compressible powder or granulation. FIG. 1B shows a side view of the upper tablet punch with a PTFE punch cup 4 on the lower tablet punch.

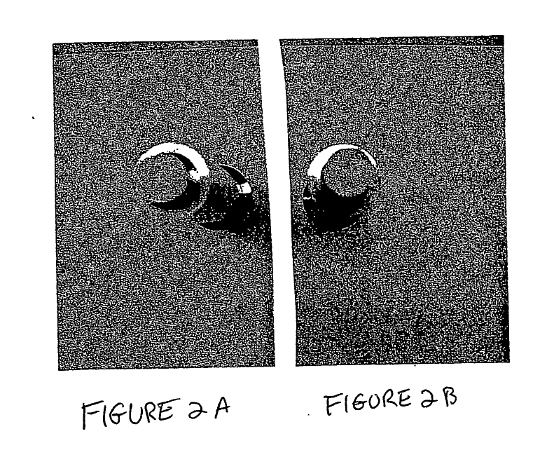

FIG. 2A shows a top view of the upper tablet punch in FIG. 1A and FIG. 2B shows a top view of FIG. 1B.

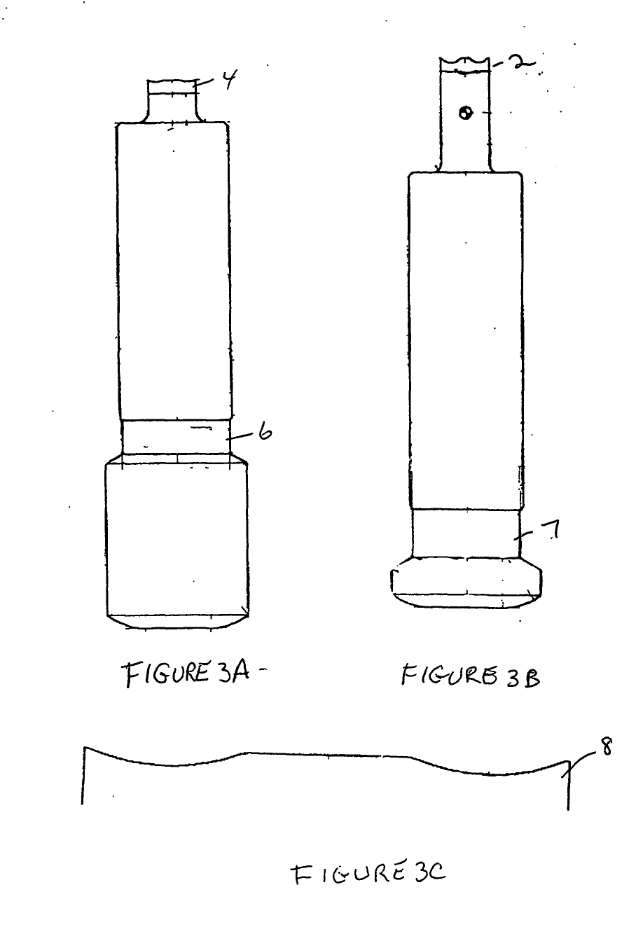

FIG. 3A is a side view of a upper tablet punch with a PTFE punch cup 4. The notched groove section 6 is adapted to engage a set screw when the punch is mounted in the tableting machine. FIG. 3A is a side view of a lower tablet punch with a PTFE punch cup 2. The notched groove section 7 is adapted to engage a set screw (not shown) when the punch is mounted in the tableting machine. FIG. 3C is an expanded section taken transversely through the center of the punch cup 2 of FIG. 3B which shows the profile of the particular punch cup illustrated in FIG. 3B.

The non-stick properties of the tablet punch are achieved by the application of a polymer layer in the cup of the tablet punch or by the application of a polymeric head. The polymer layer or head may be made from any natural or synthetic polymer or combinations of such polymers that are capable of forming a non-stick coating that can be adhered to the surface of a tablet punch cup or a head that can be fixed to the punch. In one embodiment, the non-stick coating or head comprises a fluoropolymer, preferably PTFE. In addition to PTFE, other suitable polymers which may be used to form the non-stick coating of the punch cup or head include fluorinated ethyl propylene (FEP), perfluoroalkoxy (PFA) tetrafluoroethylene perfluoromethylvinylether (MFA), tetrafluoroethylene hexafluoropropylene vinylidene fluoride (THV), ethylene tetrafluoroethylene (ETFE), ethylene chlorotrifluoroethylene (ECTFE), polyvinylidene fluoride (PVDF), polyetheretherketone (PEEK), and Ultra-High Molecular Weight Polyethylene (UHMW). The non-stick coating or head may further comprise one or more organic and/or inorganic fillers to alter properties of the coating such as wear resistance, durability, and/or strength.

The non-stick coating may be provided in uncured form, such as in the form of a monomer, oligomer, or prepolymer, and subsequently cured in situ on the surface of the punch cup. Alternatively, the non-stick coating may be provided to the punch cup in cured form such as, for example, in the form of a sheet, tape, foil, or other such form. The non-stick head maybe provided in the form of a lathed head or such other suitable form. Examples of suitable fluorinated polymers in sheet foil form include Lubriflon® PTFE foil supplied by Angst+Pfister, Zurich, Switzerland, and a self-sticking Teflon® foil supplied by Agom Tecnica SA, Bioggio, Switzerland. Examples of suitable fluorinated polymers in bars include Lubriflon® PTFE supplied by Angst+Pfister, Zurich, Switzerland.

The non-stick coating maybe applied to the punch cups in any manner suitable for obtaining good adhesion of the non-stick coating with the punch cup surface. The non-stick coating itself may have sufficient adhesive properties and may readily adhere to the surface of the punch cup without the need for additional materials or treatment. Alternatively, the use of an adhesives or the application of heat may be used to facilitate or enhance the adhesion of the non-stick coating to the surface of the punch cups. In embodiments in which the non-stick coating is provided in a flat, continuous film, such as a sheet or foil, the coating is readily applied to the punch cup by placing the film on the punch cup and removing with a sharp knife the portions of film extending beyond the periphery of the cup. Pressure is also applied to the film in the cup to enhance the bond between the film and the punch cup and to facilitate the removal of any air bubbles trapped between the film and the cup. In embodiments in which a non-stick head is used, the head is fixed to the punch by any suitable means such as, for example, one or more pins, brackets or bands. Exemplary arrangements of the tablet punch including embodiments in which the non-stick head is attached to the tablet punch by means of a pin are shown in FIGS. 1-3.

The non-stick coating and the non-stick head maybe of any suitable thickness provided that the coating and the head are sufficiently thick to provide a non-stick surface while not so thick as to adversely affect the ability of the tablet punch to produce uniformly shaped compressed forms. Preferably, the thickness of the non-stick coating is between about 0.01 microns and about 10 mm and even more preferably between about 0.1 microns and about 5 mm. Preferably, the thickness of the non-stick head is between about 1 mm and about 10 mm, and even more preferably between about 2 mm and about 5 mm.

The punch cup of the present invention may be of any conventional size and shape, and may be made from any suitable material, such as steel, provided only that the punch cup surface is capable of accommodating a non-stick layer adhered thereto. Further, the tablet punch itself may be of any suitable configuration or construction such as, for example, steel.

In operation, tablet punches of the present invention are arranged in a tablet press in one or more opposing pairs. The press further includes a die head having at least one die cavity and at least one axially-extending passageway in which the tablet punches are mounted and through which the tablet punches travel in a reciprocating movement. Suitable means are also provided for controlling the reciprocation of the punches within the passageways relative to the die head to make compressed forms. The compression of an amount of a composition by the tablet punches arranged in this manner minimizes common manufacturing problems such as sticking and capping which would otherwise occur in the absence of a non-stick coating on the punch cups or a non-stick head. These manufacturing problems are normally associated with a wide variety of compression compositions, and are particularly acute when compressing compositions that exhibit poor release characteristics such as materials that have a low melting point or have a waxy, rubbery, gluey or fatty texture such as certain pharmaceutical and other compositions.

There are numerous advantages realized by the tablet punches of the present invention. Most notably, use of tablet punches having a non-stick coating on the punch cups or a non-stick bead reduces or eliminates sticking and capping that would otherwise occur during the manufacture of compressed forms from compositions which do not contain a lubricant. Moreover, the tablet punches of the present invention obviate the need to apply a lubricating agent to the punch cups prior to compression. Through the minimization of capping and sticking, the compressed forms made by such tablet punches more readily exhibit a constant weight, shape and appearance. The use of a non-stick coating or head is suitable for a wide variety of tablet punches independent of size or shape, and suitable non-stick coating materials are readily available and easily applied to punch cup surfaces without any special tooling or procedures. If desired, the non-stick coating or head may also be readily removed from punch surface to avoid problems such as cross-contamination between products without the need for special tooling or procedures and without damage to the punch.

Another aspect of the present invention is a method for manufacturing compressed forms from a compression composition by use of a tablet press equipped with at least one pair of non-stick tablet punches. The method may be employed in the formation of pharmaceutical dosage forms, such as tablets and hard gelatin capsules, as well as in other applications in which compressed forms are desirable such as, for example, in detergents, pesticides, fungicides, food and other such applications that involve a tableting step. Suitable pharmaceutical compositions may comprise one or more active agents and, optionally, one or more pharmaceutically acceptable excipients.

The method of the present invention comprises the step of compressing an amount of a composition in a tablet press between an opposing pair of non-stick tablet punches. While the method may be applied to the compression of any composition from which compressed forms may be made, the method is particularly advantageous in the compression of compositions which do not contain a lubricating agent. In other words, these compositions consist of the active ingredients with non-lubricating excipients. Further, the non-stick coating applied to the surface of the punch cups and non-stick head permit the manufacture of compressed forms without the need to apply a lubricating agent to the surface of the punches.

In an exemplary embodiment, the method comprises the step of compressing an amount of a composition in a tablet press having a polymeric non-stick coating adhered to the punch cups of a pair of opposing tablet punches. The polymeric coating maybe a fluoropolymer, such as PTFE, or any other polymer or fluoropolymer which is capable of forming a non-stick coating 1 and of adhering to the surface of the punch cups as discussed above.

In an alternative exemplary embodiment, the method comprises the step of compressing an amount of a composition in a tablet press having a polymeric non-stick head fixed to the tips of opposing tablet punches. The polymeric head may be a fluoropolymer, such as PTFE, or any other polymer or fluoropolymer which is capable of forming a non-stick head for a tablet punch and of adhering to the tip of a tablet punch as discussed above.

Although the present invention is described in conjunction with a single punch press, the invention may be incorporated into any of a variety of conventional presses in which a plurality of tablets are produced simultaneously. Moreover, the operation of the press may include various additional steps such as pre-compression of the compression composition or other common processing steps.

The invention is further illustrated by the following non-limiting examples:

### EXAMPLE 1

An auto-adhesive PTFE film sold under the trademark Lubriflon® (code 01.1025.1015), having a thickness of approximately 0.15 mm is applied to the cups of two tablet punches which have been warmed to 60° C. for about 5 minutes by means of heated air. The quantity of film extending beyond the periphery of the cup is removed with a sharp knife. Pressure is applied to the film in the cup to remove any air bubbles trapped between the liner and the cup.

### EXAMPLE 2

An auto-adhesive PTFE film sold under the trademark Lubriflon® (code 01.1025.1050), with a thickness of approximately 0.5 mm is applied to the cups of two tablet punches which have been warmed to 60° C. for about 5 minutes by means of heated air. The quantity of film extending beyond the periphery of the cup is removed with a sharp knife. Pressure is applied to the film in the cup to remove any air bubbles trapped between the liner and the cup.

### EXAMPLE 3

An auto-adhesive PTFE film sold under the trademark Lubriflon® (code 01.1025.1100), with a thickness of approximately 1.0 mm is applied to the cups of two tablet punches which have been warmed to 60° C. for about 5 minutes by means of heated air. The quantity of film extending beyond the periphery of the cup is removed with a sharp knife. Pressure is applied to the film in the cup to remove any air bubbles trapped between the liner and the cup.

### EXAMPLE 4

An auto-adhesive PTFE film supplied by Agom Tecnica with a thickness of about 0.25 mm is applied to the cups of two tablet punches which have been warmed to 60° C. for about 5 minutes by means of heated air. The quantity of film extending beyond the periphery of the cup is removed with a sharp knife. Pressure is applied to the film in the cup to remove any air bubbles trapped between the liner and the cup.

### EXAMPLE 5

A mixture comprising 300 g S-ibuprofen, 696 g lactose, 200 g Avicel, 1 g silicon dioxide, and 3 g of magnesium stearate is obtained by direct blending of the ingredients in a cube mixer for about 20 minutes. The mixture is compressed into tablets on a rotating tableting machine Rochi AR 18 equipped with biconcave stainless steel chromium-coated punches, having a diameter of 14 mm. Tablets manufactured in this manner were of poor quality due to excessive sticking.

### EXAMPLE 6

The mixture of Example 5 is compressed on the tableting machine of Example 5 equipped with coated tablet punches as described in Example 1. Tablets produced by such tablet punches are formed without sticking or capping and are flat, round and regular with a smooth surface, each 10 mm in diameter and 1200 mg in weight and within the weight variation limits as required by the US Pharmacopoeia.

### EXAMPLE 7

In a fluid bed granulator, 700 g mannitol, 433 g sorbitol, and 40 g citric acid are granulated together with a water solution containing 7 g PVP K 25. To the dried granulate is added 100 g of ubidecarenone (coenzyme QIO), 8 g orange flavor, and 12 g magnesium stearate. The mixture is blended in a cube mixer for about 15 minutes in order to reach homogeneity. The resulting mixture is compressed into fast-melt tablets on a rotating tableting machine using a toroidal steel chromium-coated punch, 16 mm in diameter. The tablets contain 100 mg micronized ubidecarenone, weigh 1300 mg each, and demonstrate poor mechanical characteristics due to sticking of the mixture to the punch cups.

### EXAMPLE 8

The mixture of Example 7 is compressed on the tableting machine of Example 7 equipped with coated tablet punches as described in Example 1. Tablets produced by such tablet punches are formed without sticking or capping and are flat, round and regular with a smooth surface, 16 mm in diameter, and within the weight variation limits as required by the US Pharmacopoeia.

### EXAMPLE 9

139.62 g mannitol, 113.5 g sorbitol, and 10.90 g citric acid are granulated together with a water solution containing 5.45 g of PEG 6000. To the dried granulate is added 4.58 g tizanidine HCl, 9 g grapefruit flavor, 14.4 g aspartame, and 3 g magnesium stearate. The mixture is blended for about 15 minutes in order to reach homogeneity. The resulting mixture is compressed into fast melt tablets on a rotating tableting machine using a toroidal steel, chromium-coated punch, 10 mm in diameter weighing 300 mg each and containing 4 mg micronized tizanidine HCl. The resulting tablets showed poor mechanical characteristics due to sticking of the mixture to the punch cups.

### EXAMPLE 10

The mixture of Example 9 is compressed on the tableting machine of Example 9 equipped with coated tablet punches as described in Example 2. Tablets produced by such tablet punches are formed without sticking or capping and are toroidal, beveled-edge tablets, 10 mm in diameter and 300 mg in weight and within the weight variation limits as required by the US Pharmacopoeia.

### EXAMPLE 11

2000 g ibuprofen, 6150 g sodium bicarbonate, and 1500 g sodium carbonate are granulated together in a fluid bed granulator with a water solution containing 25 g of sodium docusate. The granulate (Granulate A) is then dried. 4000 g citric acid, 250 g sodium saccharine, 2846 g sodium bicarbonate, and 250 g aspartame are granulated with a water solution in a fluid bed granulator. The granulate is then partially dried. To the granulate is added 2300 g trisodium citrate and 1428 g sodium bicarbonate. The granulate (Granulate B) is then dried. Granulates A and B are then blended together for about 15 minutes with 801 g of berry flavor and 450 g of sodium benzoate to homogeneity. The resulting mixture is compressed on a rotating tableting machine using a flat punch of 18 mm in diameter coated with a polyethylene liner to obtain effervescent tablets containing 200 mg ibuprofen and weighing 2400 mg each. The resulting tablets showed poor mechanical characteristics when compressed on a rotating tableting machine due to sticking of the mixture to the punch cups.

### EXAMPLE 12

The mixture of Example 11 is compressed on the tableting machine of Example 11 equipped with coated tablet punches as described in Example 3. Tablets produced by such tablet punches are formed without sticking or capping and are flat-faced, beveled-edge tablets, 18 mm in diameter, and 2.4 g in weight within in the limits required by the US Pharmacopoeia.

### EXAMPLE 13

775.2 g mannitol, 496.4 g sorbitol, 54.4 g citric acid and 17 g aspartame are granulated together with water solution containing 17.0 g PEG 6000. To the dried granulate is added 100 g lovastatine, 20 g of lemon flavor, g and 20 g of magnesium stearate. The mixture is blended for about 15 minutes to homogeneity. The resulting mixture is compressed on a rotating tableting machine using a toroidal steel, chromium-coated punch, 8 mm in diameter, to obtain fast melt tablets containing 10 mg of lovastatine and weighing 150 mg each. The tablets showed poor mechanical characteristics due to sticking of the mixture to the punch cups.

### EXAMPLE 14

The mixture of Example 13 is compressed on the tableting machine of Example 13 equipped with coated tablet punches as described in Example 1. Tablets produced by such tablet punches are formed without sticking or capping and are flat-faced, beveled-edge tablets, 8 mm in diameter, and 150 mg in weight within the limits required by the US Pharmacopoeia.

### EXAMPLE 15

1168 g mannitol, 292 g of xylitol, 130 g citric acid, 50 mg aspartame, and 115 g orange flavor are granulated together in a fluid bed granulator with a water solution containing 200 g PEG 6000. To the dried granulate is added 1000 g of micronized nimesulide, 1000 g sorbitol, 20 g Aerosil and 25 g of magnesium stearate. The mixture is blended for about 15 minutes to homogeneity. The resulting mixture is compressed on a rotating tableting machine using a toroidal steel, chromium-coated punch, 12 mm in diameter, to obtain fast melt tablets containing mg 100 of nimesulide and weighing 400 mg each. The resulting tablets showed poor mechanical characteristics due to sticking of the mixture to the punch cups.

### EXAMPLE 16

The mixture of Example 15 is compressed on the tableting machine of Example 15 equipped with coated tablet punches as described in Example 2. Tablets produced by such tablet punches are formed without sticking or capping and are toroidal, beveled-edge tablets, 12 mm in diameter and 400 mg in weight within the limits as required by the US Pharmacopoeia.

### EXAMPLE 17

A mixture of 2000 g of S-ibuprofen, 1 g of lactose, 2000 g dried maize starch, 10 g of silicon dioxide, and 30 g of magnesium stearate is filled into hard gelatin capsules using a Zanasi capsuling machine equipped with flat stainless steel, chromium-coated punches. The resultant capsules are not uniform in weight due to sticking of the mixture to the punches.

### EXAMPLE 18

The mixture of Example 17 is compressed on the same capsuling machine as in Example 17 equipped with coated tablet punches as described in Example 1. The hard gelatin capsules are formed without sticking and weigh 1200 mg each with a variation within the limits as required by the US Pharmacopoeia.

### EXAMPLE 19

1271.6 g lactose, 17 g aspartame, and 54.4 g sodium carboxymethyl cellulose are granulated in a fluid bed granulator, together with a water solution containing 17 g of PEG. To the dried granulate is added 20 g magnesium stearate, 20 g lemon flavor, and 100 g enalapril maleate. The mixture is blended for about 15 minutes to homogeneity. The resulting mixture is compressed on a rotating tableting machine using a Martensitic steel, flat, round, edge-beveled punch 8 mm in diameter, to obtain chewable tablets containing 10 mg of enalapril maleate, and weighing 150 mg each.

### EXAMPLE 20

The mixture of Example 19 is compressed on the same rotating tableting machine of Example 19 equipped with coated tablet punches as described in Example 4. The chewable tablets are formed without sticking and weigh 150 mg each with a variation within the limits as required by the US Pharmacopoeia.

### EXAMPLE 21

333.40 g Aerosil, 833.4 g lycopene 18% (solution) and 500 g mannitol are blended together to obtain a dry fine powder. 7695.3 g sucrose fine powder and 331.9 g citric acid are granulated in a fluid bed granulator, together with a water solution containing 166 g PEG 6000. The lycopene-containing powder and the granulate are added with 60 g magnesium stearate and 80 g of wild berry flavor. The mixture is blended for about 15 minutes to homogeneity. The resulting mixture is compressed on a rotating tableting machine using biconcave stainless steel, chromium-coated punches, 16 mm in diameter, to obtain chewable tablets containing 15 mg lycopene and weighing 1000 mg each.

### EXAMPLE 22

The mixture of Example 21 is compressed on the same rotating tableting machine of Example 20 equipped with coated tablet punches as described in Example 4. The chewable tablets are formed without sticking and weigh 1000 mg each with a variation within the limits as required by the US Pharmacopoeia.

### EXAMPLE 23

1293.6 g mannitol, 839.4 g sorbitol, and 92.0 g citric acid are granulated together in a fluid bed granulator, with a water solution containing g 46.0 g PEG 6000. To the dried granulate is added 100 g micronized loratadine, 12.6 g Aerosil A 200, 72.0 g lemon flavor, 28.8 g aspartame, and 15.7 g magnesium stearate. The mixture is blended for about 15 minutes to homogeneity. The resulting mixture is compressed on a rotating tableting machine using a toroidal steel, chromium-coated punch, 10 mm in diameter, to obtain fast-melt tablets containing 10 mg loratadine and weighing 250 mg each. The resulting tablets showed poor mechanical characteristics due to sticking of the mixture to the punches.

### EXAMPLE 24

The mixture of Example 23 is compressed on the same rotating tableting machine of Example 23 equipped with coated tablet punches as described in Example 2. The fast-melt tablets are formed without sticking and weigh 2500 mg each with a variation within the limits as required by the US Pharmacopoeia.

### EXAMPLE 25

A PTFE head sold under the trademark Lubriflon®, having a thickness of approximately 3.5 mm is applied with a horizontal pin to the tablet punches of a Rochi AR 18 rotating tableting machine.

### EXAMPLE 26

A mixture comprising 300 g S-ibuprofen, 696 g lactose, 200 g Avicel, 1 g silicon dioxide, and 3 g of magnesium stearate is obtained by direct blending of the ingredients in a cube mixer for about 20 minutes. The mixture is compressed into tablets on a rotating tableting machine Rochi AR 18 equipped with biconcave stainless steel, chromium coated punches, having a diameter of 14 mm. Tablets manufactured in this manner were of poor quality due to excessive sticking.

### EXAMPLE 27

The mixture of Example 26 is compressed on the tableting machine of Example 26 equipped with punches having non-stick lathed heads as described in Example 25. Tablets produced by such tablet punches are formed without sticking or capping and are flat, round and regular with a smooth surface, each 10 mm in diameter and 1200 mg in weight and within the weight variation limits as required by the US Pharmacopoeia.

### EXAMPLE 28

In a fluid bed granulator, 700 g mannitol, 433 g sorbitol, and 40 g citric acid are granulated together with a water solution containing 7 g PVP K 25. To the dried granulate is added 100 g of ubidecarenone (coenzyme QIO), 8 g orange flavor, and 12 g magnesium stearate. The mixture is blended in a cube mixer for about 15 minutes in order to reach homogeneity. The resulting mixture is compressed into fast-melt tablets on a rotating tableting machine using a 1 toroidal steel, chromium-coated punch 16 mm in diameter. The tablets contain 100 mg micronized ubidecarenone, weigh 1300 mg each, and demonstrate poor mechanical characteristics due to sticking of the mixture to the punch cups.

### EXAMPLE 29

The mixture of Example 28 is compressed on the tableting machine of Example 28 equipped with punches with non-stick lathed heads as described in Example 25. Tablets produced by such tablet punches are formed without sticking or capping and are flat, round and regular with a smooth surface, 16 mm in diameter, and within the weight variation limits as required by the US Pharmacopoeia.

### EXAMPLE 30

139.62 g mannitol, 113.5 g sorbitol, and 10.90 g citric acid are granulated together with a water solution containing 5.45 g of PEG 6000. To the dried granulate is added 4.58 g tizanidine HCl, 9 g grapefruit flavor, 14.4 g aspartame, and 3 g magnesium stearate. The mixture is blended for about 15 minutes in order to reach homogeneity. The resulting mixture is compressed into fast melt tablets on a rotating tableting machine using a toroidal steel, chromium-coated punch, 10 mm in diameter weighing 300 mg each and containing 4 mg micronized tizanidine HCl. The resulting tablets showed poor mechanical characteristics due to sticking of the mixture to the punch cups.

### EXAMPLE 31

The mixture of Example 30 is compressed on the tableting machine of Example 30 equipped with punches with non-stick lathed heads as described in Example 25. Tablets produced by such tablet punches are formed without sticking or capping and are toroidal, beveled-edge tablets, 10 mm in diameter and 300 mg in weight and within the weight variation limits as required by the US Pharmacopoeia.

### EXAMPLE 32

2000 g ibuprofen, 6150 g sodium bicarbonate, and 1500 g sodium carbonate are granulated together in a fluid bed granulator with a water solution containing 25 g of sodium docusate. The granulate (Granulate A) is then dried. 4000 g citric acid, 250 g sodium saccharine, 2846 g sodium bicarbonate, and 250 g aspartame are granulated with a water solution in a fluid bed granulator. The granulate is then partially dried. To the granulate is added 2300 g ‘trisodium citrate’ and 1428 g sodium bicarbonate. The granulate (Granulate B) is then dried.

Granulates A and B are then blended together for about 15 minutes with 801 g of berry flavor and 450 g of sodium benzoate to homogeneity. The resulting mixture is compressed on a rotating tableting machine using a flat punch, 18 mm in diameter and coated with a polyethylene liner to obtain effervescent tablets containing 200 mg ibuprofen and weighing 2400 mg each. The resulting tablets showed poor mechanical characteristics when compressed on a rotating tableting machine due to sticking of the mixture to the punch cups.

### EXAMPLE 33

The mixture of Example 32 is compressed on the tableting machine of Example 32 equipped with punches with non-stick lathed heads as described in Example 25. Tablets produced by such tablet punches are formed without sticking or capping and are flat-faced, beveled-edge tablets, 18 mm in diameter, and 2.4 g in weight within in the limits required by the US Pharmacopoeia.

### EXAMPLE 34

775.2 g mannitol, 496.4 g sorbitol, 54.4 g citric acid and 17 g aspartame are granulated together with water solution containing 17.0 g PEG 6000. To the dried granulate is added 100 g lovastatine, 20 g of lemon flavor, g and 20 g of magnesium stearate. The mixture is blended for about 15 minutes to homogeneity. The resulting mixture is compressed on a rotating tableting machine using a toroidal steel, chromium-plated punch, 8 mm in diameter, to obtain fast melt tablets containing 10 mg of lovastatine and weighing 150 mg each. The tablets showed poor mechanical characteristics due to sticking of the mixture to the punch cups.

### EXAMPLE 35

The mixture of Example 34 is compressed on the tableting machine of Example 34 equipped with punches with non-stick lathed heads as described in Example 25. Tablets produced by such tablet punches are formed without sticking or capping and are flat-faced, beveled-edge tablets, 8 mm in diameter, and 150 mg in weight within the limits required by the US Pharmacopoeia

### EXAMPLE 36

1168 g mannitol, 292 g of xylitol, 130 g citric acid, 50 mg aspartame, and 115 g orange flavor are granulated together in a fluid bed granulator with a water solution containing 200 g PEG 6000. To the dried granulate is added 1000 g of micronized nimesulide, 1000 g sorbitol, 20 g Aerosil and 25 g of magnesium stearate. The mixture is blended for about 15 minutes to homogeneity. The resulting mixture is compressed on a rotating tableting machine using a toroidal steel, chromium-plated punch, 12 mm in diameter, to obtain fast melt tablets containing mg 100 of nimesulide and weighing 400 mg each. The resulting tablets showed poor mechanical characteristics due to sticking of the mixture to the punch cups.

### EXAMPLE 37

The mixture of Example 36 is compressed on the tableting machine of Example 36 equipped with punches with non-stick lathed head as described in Example 25. Tablets produced by such tablet punches are formed without sticking or capping and are toroidal, beveled-edge tablets, 12 mm in diameter and 400 mg in weight within the limits as required by the US Pharmacopoeia.

### EXAMPLE 38

A mixture of 2000 g of S-ibuprofen, 1 g of lactose, 2000 g dried maize starch, 10 g of silicon dioxide, and 30 g of magnesium stearate is filled into hard gelatin capsules using a Zanasi capsuling machine equipped with flat stainless steel, chromium-plated punches. The resultant capsules are not uniform in weight due to sticking of the mixture to the punches.

### EXAMPLE 39

The mixture of Example 38 is compressed on the same capsuling machine as in Example 38 equipped with punches with non-stick lathed heads as described in Example 25. The bard gelatin capsules are formed without sticking and weigh 1200 mg each with a variation within the limits as required by the US Pharmacopoeia.

### EXAMPLE 40

1271.6 g lactose, 17 g aspartame, and 54.4 g sodium carboxymethyl cellulose are granulated in a fluid bed granulator, together with a water solution containing 17 g of PEG. To the dried granulate is added 20 g magnesium stearate, 20 g lemon flavor, and 100 g enalapril maleate. The mixture is blended for about 15 minutes to homogeneity. The resulting mixture is compressed on a rotating tableting machine using a Martensitic steel, flat, round, edge-beveled punch, 8 mm in diameter, to obtain chewable tablets containing 10 mg of enalapril maleate, and weighing 150 mg each. The resultant capsules are not uniform in weight due to sticking of the mixture to the punches.

### EXAMPLE 41

The mixture of Example 40 is compressed on the same rotating tableting machine of Example 40 equipped with punches with non-stick lathed heads as described in Example 25. The chewable tablets are formed without sticking and weigh 150 mg each with a variation within the limits as required by the US Pharmacopoeia.

### EXAMPLE 42

333.40 g Aerosil, 833.4 g lycopene 18% (solution) and 500 g manxiitol are blended together to obtain a dry fine powder. 7695.3 g sucrose fine powder and 331.9 g citric acid are granulated in a fluid bed granulator, together with a water solution containing 166 g PEG 6000. The lycopene-containing powder and the granulate are added with 60 g magnesium stearate and 80 g of wild berry flavor. The mixture is blended for about 15 minutes to homogeneity: The resulting mixture is compressed on a rotating tableting machine using biconcave, stainless steel, chromium-plated punches, 16 mm in diameter, to obtain chewable tablets containing 15 mg lycopene and weighing 1000 mg each. The resultant capsules are not uniform in weight due to sticking of the mixture to the punches.

### EXAMPLE 43

The mixture of Example 42 is compressed on the same rotating tableting machine of Example 42 equipped with punches with non-stick lathed heads as described in Example 25. The chewable tablets are formed without sticking and weigh 1000 mg each with a variation within the limits as required by the US Pharmacopoeia.

### EXAMPLE 44

1293.6 g mannitol, 839.4 g sorbitol, and 92.0 g citric acid are granulated together in a fluid bed granulator, with a water solution containing g 46.0 g PEG 6000. To the dried granulate is added 100 g micronized loratadine, 12.6 g Aerosil A 200, 72.0 g lemon flavor, 28.8 g aspartame, and 15.7 g magnesium stearate. The mixture is blended for about 15 minutes to homogeneity. The resulting mixture is compressed on a rotating tableting machine using a toroidal steel, chromium-plated punch, 10 mm in diameter, to obtain fast-melt tablets containing 10 mg loratadine and weighing 250 mg each. The resulting tablets showed poor mechanical characteristics due to sticking of the mixture to the punches.

### EXAMPLE 45

The mixture of Example 44 is compressed on the same rotating tableting machine of Example 44 equipped with punches with non-stick lathed head as described in Example 25. The fast-melt tablets are formed without sticking and weigh 2500 mg each with a variation within the limits as required by the US Pharmacopoeia.

I claim:

1. A tablet punch comprising a punch cup having a non-stick polymeric coating applied thereto.

2. The tablet punch of claim 1 wherein the non-stick polymeric coating comprises a fluoropolymer.

3. The tablet punch of claim 1 wherein the non-stick polymeric coating comprises polytetrafluoroethylene.

4. The tablet punch of claim 3 wherein the polymeric coating has a thickness between about 0.01 microns and about 10 mm.

5. A tablet punch comprising a punch tip having a non-stick polymeric lathed head affixed thereto.

6. The tablet punch of claim 5 wherein the head comprises a fluoropolymer.

7. The tablet punch of claim 5 wherein the head comprises polytetrafluoroethylene.

8. The tablet punch of claim 5 wherein the head has a thickness between about 0.1 mm and about 5 mm.

9. A method for manufacturing compressed forms comprising the step of compressing an amount of a composition in a tablet press between an opposing pair of tablet punches where the punch cups of said punches have a non-stick polymeric coating.

10. The method of claim 9 wherein the non-stick polymeric coating comprises a fluoropolymer.

11. The method of claim 9 wherein the non-stick polymeric coating comprises polytetrafluoroethylene.

12. A method for manufacturing compressed forms comprising the step of compressing an amount of a composition in a tablet press between an opposing pair of the non-stick tablet punches of claim 5.

13. The method of claim 12 wherein the non-stick polymeric coating comprises a fluoropolymer.

14. The method of claim 12 wherein the non-stick polymeric coating comprises polytetrafluoroethylene.

15. The method of claim 9 wherein the composition comprises a pharmaceutical composition.

16. The method of claim 15 wherein the pharmaceutical composition does not include a lubricating agent.

17. The method of claim 12 wherein the composition comprises a pharmaceutical composition.

18. The method of claim 17 wherein the pharmaceutical composition does not include a lubricating agent.

19. A compressed tablet which consists of an active ingredient and non-lubricating tablet excipients.
